(19) United States
(12) Patent Application Publication
(10) Pub. No.: US 2013/0178774 A1
(43) Pub. Date: Jul. 11, 2013

(54) MULTI-STRAP LUMBAR SUPPORT DEVICE

(71) Applicant: Victor HAYES, Odessa, FL (US)

(72) Inventor: Victor HAYES, Odessa, FL (US)

(21) Appl. No.: 13/727,122

(22) Filed: Dec. 26, 2012

Related U.S. Application Data
(60) Provisional application No. 61/580,284, filed on Dec. 26, 2011.

(51) Int. Cl. A61F 5/02

(52) U.S. Cl. CPC A61F 5/028
USPC 602/19

(57) ABSTRACT

Provided are adjustable, multi-strap lumbar support devices that may be used to provide lumbar and/or belly support for mammals having belly weight. According to non-limiting example embodiments, support or brace devices provided herein may be worn by pregnant women at various stages throughout a woman's pregnancy, and may be adjusted to naturally contour/conform to the woman's anatomy, which changes throughout pregnancy. Also provided herein are methods of preventing and/or reducing back pain in a mammal that include applying the lumbar support to a mammal as directed. Further provided are kits that include at least one multi-strap lumbar support device provided herein, which kit may optionally include instructions for proper application and/or adjustment of the multi-strap, lumbar support device.

### CROSS REFERENCE TO RELATED APPLICATION

This application claims the benefit of U.S. provisional application No. 61/580,284, filed on Dec. 26, 2011, the contents of which are hereby incorporated by reference in their entirety.

### TECHNICAL FIELD

Present embodiments relate generally to multi-strap, lumbar support devices and methods of treating and/or preventing back pain in a person, due to for example pregnancy or other belly weight in the person. Further provided are kits including such lumbar support devices.

### BACKGROUND ART

Back pain is a common complaint during pregnancy. Typically a woman's body will gain between 15-40 pounds. Pregnancy causes the body's hormones to fluctuate causing many physiologic changes throughout the body. The key hormones that dramatically fluctuate during pregnancy are estrogen, progesterone and relaxin. Fluctuations in both estrogen and progesterone are necessary for a woman to become pregnant and for an expecting mother to carry her baby to term. During pregnancy high progesterone levels function to maintain a healthy internal environment for the baby. However, as the progesterone and relaxin levels increase during pregnancy, joints, ligaments, tendons and tissues throughout the body relax and stretch.

Once the joints and ligaments relax, the lower back becomes more flexible and the body naturally changes the spinal alignment to compensate for the excess “belly weight” throughout pregnancy. However, the added “belly weight” is like carrying a heavy object in front of the lower pelvis. This added weight during pregnancy changes the center of gravity, so one's body compensates by increasing their lower back's natural curvature called Lordosis. The body's center of gravity is a single point at which gravity exerts its downward force. For one to balance and stand without falling over, a person's center of gravity must be balanced by coordinating and using their different muscle groups (especially core muscles). Lordosis is a backward curvature of spine and helps to counter balance the change in the center of gravity.

In objects with an irregular or changing shape, as in the human body during pregnancy, the center of gravity changes throughout different stages of pregnancy. Typically in a normal, healthy, standing individual, the center of gravity is located about a icm behind the junction of the lumbar spine and sacrum. However, during pregnancy, the excess “belly weight” shifts the center of gravity forward several inches

During pregnancy the combination of the increased “belly” weight, increase in spinal flexibility, change in the center of gravity, and altered lumbar lordosis create a delicate equilibrium. Any additional activities or postural changes that create undo stress on the lumbar spine can lead to imbalance in the equilibrium. Ultimately all of these changes and this imbalance cause muscle fatigue. This muscle fatigue is a common cause of pregnancy related back pain and can develop into a more serious spinal disorder if untreated. Even in fit women lower back pain is a painful reality that will affect 50-80 percent of all pregnant women.

Typical treatments for back pain include administration of anti-inflammatories and other medications/treatments, which must be avoided during pregnancy to avoid any harmful affect to the baby.

### SUMMARY

Provided herein are fully adjustable, multi-strap lumbar support devices that may be used to provide support for persons having excess belly weight, such as for pregnant women, persons with ascites, overweight persons with excess belly weight, etc. According to non-limiting example embodiments, support or brace devices provided herein may be used throughout a woman's pregnancy, and may be adjusted to naturally contour/conform to the woman's anatomy, which changes throughout pregnancy.

The present support devices are effective at decreasing the stresses on the spine, and improving posture/alignment during activities of daily living.

Also provided herein are methods of preventing and/or reducing back pain in a person that include applying a lumbar support device provided herein to a person.

Further provided are kits that include at least one multi-strap lumbar support device provided herein, which kit may optionally include instructions for proper application and/or adjustment of the multi-strap lumbar support device.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. herein is provided for the purpose of illustration and description only and is by no means intended to define the limits the invention.

### BRIEF DESCRIPTION OF THE DRAWINGS

Non-limiting example embodiments described herein, with reference to the following accompanying Figures.

FIG. 1 depicts a front/anterior view of an example modular multi-strap lumbar support device (1) provided herein. This view depicts, inter alia, top and bottom understraps (2 and 3, respectively) and top and bottom overstraps (4 and 5, respectively) in an open position and not closed over a wearer of the device. Straps may be removable and may e.g., clip, snap, hook, and/or zipper on or off.

FIG. 2 depicts a front/anterior view of the example modular multi-strap lumbar support device of FIG. 1. This view depicts, inter alma, the top and bottom understraps in a closed position across the front of the device, for example with respect to a wearer of the present support device, and the top and bottom overstraps (4 and 5, respectively) in an open position and not yet wrapped over the top of the top and bottom understraps.

FIG. 3 depicts a front/anterior view of the example modular multi-strap lumbar support device of FIGS. 1 and 2. This view depicts the top and bottom understraps in a closed position across the front of the device, with the top and bottom overstraps (4 and 5, respectively) in a closed position over the top of the respective top and bottom understraps.

FIG. 4 depicts a back/posterior view of the example modular multi-strap lumbar support device of FIGS. 1-3. As shown in FIG. 4, a back side of the brace may include two or more panels (6 and 7), which may be connected to one another e.g., by cinch strings (8). A tensioner reel 13 (adjuster) may be provided to allow for the cinch strings to be loosened or tightened. This tensioner reel 13 may be attached to a posterior panel 7. Snaps or clips 15 (or zippers or other attachment) may be present to attach the removable straps (upper and lower). The strings are anchored to the tension reel 13 and fixed to panel 6 at a fixed equal (or approximately equal) distance. The spandex/elastic material 12 will be sewed into the posterior panels 6 and 7 directly. These embodiments of the support may therefore be slid on over one's head to wear. Other embodiments may include a zipper 16 or other attachment that permits one to put on the brace without having to slip it over the wearer's head or step into it.

FIG. 5 depicts a back/posterior view of the example modular multi-strap lumbar support device of FIGS. 1-4. As shown in FIG. 5, the understraps and overstraps may be detachable from other portions of the support device, e.g., from one or more of the panels.

FIG. 6 depicts a front/anterior view of another example multi-strap lumbar support device provided herein. This view depicts, inter alia, top and bottom understraps (102 and 103) across the front of the device.

FIG. 7 depicts a front/anterior view of the example multi-strap lumbar support device of FIG. 6. This view depicts, inter alia, the top and bottom overstraps (104 and 105) across the front of the device.

FIG. 8 depicts a front/anterior view of the example multi-strap lumbar support device of FIGS. 6-7. This view depicts, inter glia, the top and bottom understraps (102 and 103) and the top and bottom overstraps (104 and 105) in an open position and not closed over a wearer of the device.

FIG. 9 depicts a front/anterior view of the example multi-strap lumbar support device of FIGS. 6-8. This view depicts the top and bottom understraps (102 and 103) in a closed position across the front of the device, with the top and bottom overstraps (104 and 105) in a closed position over the top of the respective top and bottom understraps.

FIG. 10 depicts a back/posterior view of the example multi-strap lumbar support device of FIGS. 6-9. As shown in FIG. 10, multiple pullstrings (e.g., 106 and 107) from multiple channels (e.g., 108 and 109) may optionally be provided through upper and lower overstraps on the anterior side of the device, and may be consolidated into one or more pullstrings through for example, a single channel 110. The consolidated pullstrings may optionally attach to a pullstring attachment 111, such that a user/wearer of the device may pull the pullstrings to adjust the fit of the device.

FIG. 11 depicts a back/posterior view of the example multi-strap lumbar support device of FIGS. 6-10. As shown in FIG. 11, multiple pullstrings (e.g., 113 and 114) from multiple channels (e.g., 115 and 116) may optionally be provided through upper and lower overstraps on the anterior side of the device, and may be consolidated into one or more pullstrings through for example, a single channel 117. The consolidated pullstrings may attach to a pullstring attachment 112, such that a user/wearer of the device may pull the pullstrings to adjust the fit of the device.

FIG. 12 depicts a right side view of the example multi-strap lumbar support device of FIGS. 6-11 in position over a human. As shown in FIG. 12, the optional pullstrings may be spread over various positions of the human's belly, but are consolidated toward the back and may be adjusted by pulling on a pullstring attachment on the other side of the human. That is, FIG. 12 depicts pullstrings (e.g., 113 and 114) throughout the right side (understraps) of the device, but the shown consolidated pullstring 110, which attaches to pullstring attachment 111 may be pulled or loosened to adjust the fit of the pullstrings in the opposite/left side (overstraps) of the device.

FIG. 13 depicts a right side view of the example multi-strap lumbar support device of FIGS. 6-12 in position over a human. As shown in FIG. 13, the optional pullstrings may be spread over various positions of the human's belly, but are consolidated toward the back and may be adjusted by pulling on a pullstring attachment on the other side of the human. That is, FIG. 13 depicts pullstrings (e.g., 106 and 107 throughout the left side (overstraps) of the device, but the shown consolidated pullstring 117, which attaches to pullstring attachment 112 may be pulled or loosened to adjust the fit of the pullstrings in the opposite/right side (understraps) of the device.

### DETAILED DESCRIPTION

Generally provided herein are support devices that may be useful for prevention and treatment of back pain in a person. By way of non-limiting example, the present embodiments may include devices for prevention of back pain in pregnant female humans or other persons that may for example, have excess belly weight, or persons that may have for example, Ascites.

Also provided herein are kits that include the present devices, and methods of preventing and/or reducing back pain in a person that include applying the present devices to a person in need thereof. A person in need of the present devices may include for example, pregnant female humans, persons having ascites, and/or persons having excess weight or bulk around the belly area.

Additional aspects, advantages and/or other features of example embodiments will become apparent in view of the following detailed description, taken in conjunction with the accompanying drawings. It should be apparent to those skilled in the art that the described embodiments provided herein are merely exemplary and illustrative and are not limiting. Numerous embodiments of modifications thereof are contemplated as falling within the scope of this disclosure and equivalents thereto.

Any references mentioned in this specification are indicative of the level of those skilled in the art to which the invention pertains. Any references herein are incorporated by reference to the same extent as if each was specifically and individually indicated as having been incorporated by reference in its entirety.

Unless otherwise noted, technical terms are used according to conventional usage.

As used herein, “a” or “an” may mean one or more. Also as used herein in the claim(s), when used in conjunction with the word “comprising”, the words “a” or “an” may mean one or more than one. As used herein “another” may mean at least a second or more. Furthermore, unless otherwise required by context, singular terms include pluralities and plural terms include the singular.

Provided herein are adjustable, multi-strap lumbar support devices. Example embodiments include lumbar supports having at least one understrap, at least one overstrap, and at least one posterior panel.

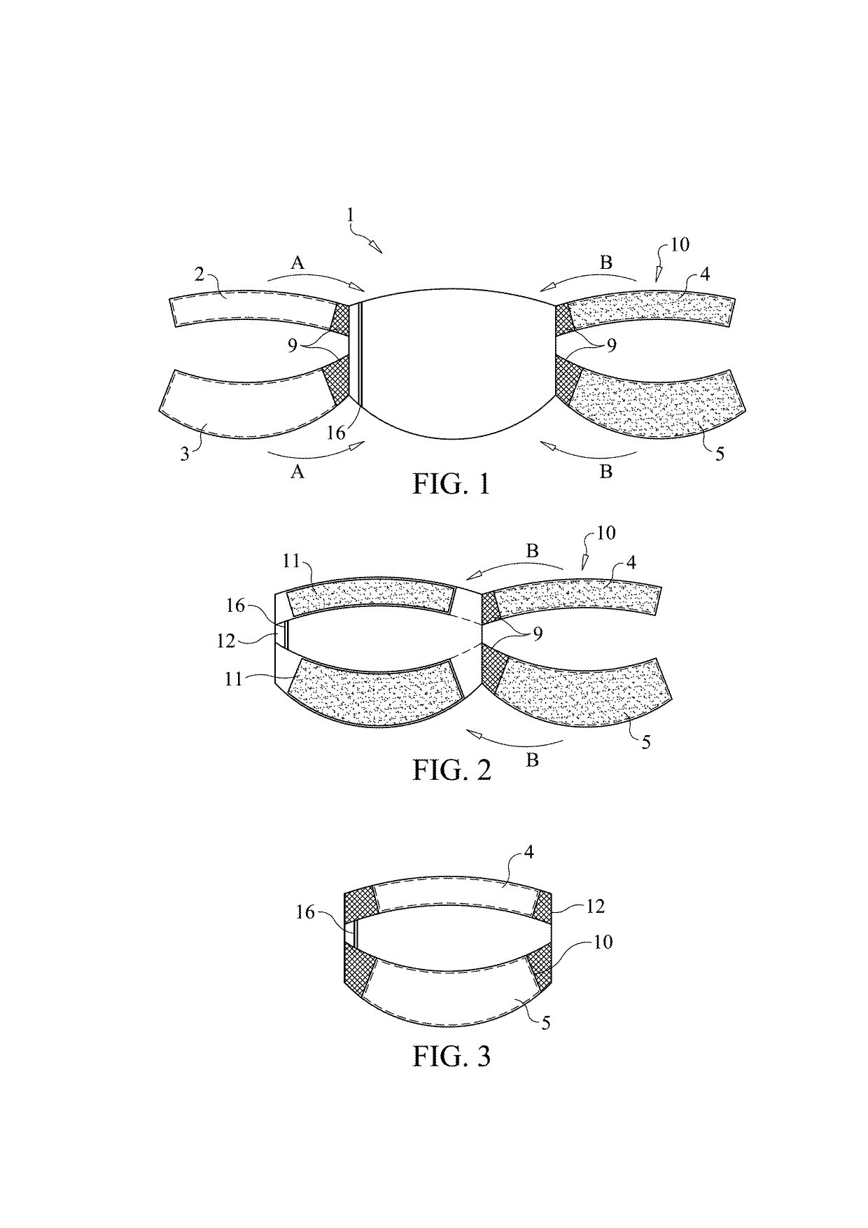

According to example embodiments, a modular lumbar support device is provided having at least one understrap (2, 3) and at least one overstrap (4, 5); at least first and second posterior panels (7, 6), the at least one understrap being detachably attached to the first posterior panel, and at least one overstrap being detachably attached to the second posterior panel; wherein the first and second posterior panels are connected to one another; and wherein said at least one understrap and said at least one overstrap are adapted to be attachable to each other when said understrap and said overstrap are attached to the device and in a closed position. The at least one understrap and the at least one overstrap close across a front of a wearer of the device when the understrap(s) and overstrap(s) are attached to the device and in a closed position. According to non-limiting example embodiments, the device may include at least two understraps and at least two corresponding overstraps, for example as depicted in FIGS. 1-5.

The example devices also include and at least first and second posterior panels (7 and 6). In example embodiments, the at least one understrap (e.g., 2 and/or 3) may be attached to a first posterior panel (7), and the at least one overstrap (e.g., 4 and/or 5) may be attached to a second posterior panel (6). Snaps, clips and/or zippers or other attachments may be present to attach the removable straps.

According to example embodiments, the posteriors panels 6 and 7 may be permanently attached to one another by cinch strings, e.g., plastic strings, and these strings may be adjusted by the tension reel on the panel 7. These strings may be fixed at an equal distance on both panels 6 (directly attached 14) and 7 (attached by tension reel system). 6 and 7 components may be fixed to a spandex/elastic anterio material (12)/lining material, which may be adapted for example to wrap all the way around the belly of a wearer of the device. Because the posterior panels do not detach from one another in example embodiments, the support device in these embodiments may be pulled over the wearer's head or the wearer may step into the device to put the device on. In alternative embodiments, the anterio material/lining material/corset component may be configured such that it may be opened or loosened in one or more positions, e.g., using a zipper 16. Such configurations may allow one to more easily put the device on, e.g., by zipping a side of the device and then closing the straps.

A tensioner reel 13 (adjuster) may be provided to allow for the cinch strings to be loosened or tightened. This tensioner reel 13 may be attached to a posterior panel 7. The strings are anchored to the tension reel 13 and fixed to panel 6 at a fixed equal (or approximately equal) distance. The spandex/elastic material 12 will be sewed into the posterior panels 6 and 7 directly. These embodiments of the support may therefore have to be slid on over one's head to wear, or opened up e.g., by one or more zippers 16 or other attachments, as discussed above.

These embodiments of the present designs are novel at least because they are modular: allowing the different components to be added or subtracted as a persons' size changes, e.g., to better conform to a women's anatomic changes throughout pregnancy. One of the major concerns with other braces and back supports is that over tensioning can create excess pressure on the abdomen of the pregnant female. The present braces combine multiple safeguards to prevent this (over tensioning) while at the same time providing more contouring support by conforming to the anatomy throughout the changes a female has in each trimester pregnancy. Example safeguards to prevent over tensioning may include for example: a spandex front panel attached to the back corset component, elastic material on the detachable straps 9 (both upper and lower), and elastic strings in the corset component of the support device, which strings may be adjusted by the tension reel). The present tension reel system is also novel.

The goal of the present supports is to provide stabilization throughout each trimester of pregnancy. Initially, during the coarse of a woman's pregnancy, the corset (e.g., spandex or elastic material) component alone (without the straps attached) may be used during the first and possibly early second trimester, as the straps are typically not necessary to support the belly (which is usually not present into the second and third trimesters). However, as the belly grows throughout the second and third trimester of pregnancy, the lower straps can be clipped on, snapped on, zipped on, or otherwise attached easily to allow for better support. An upper strap will be used for example in the last days of pregnancy to avoid the brace from sliding off to the abdominal pressure on the lower strap.

The understraps and overstraps may be made of various materials, such as for example, a breathable cloth, which may include at least one stretch material, such as spandex. Other suitable materials may be determined by those skilled in the art. The straps may be made of a single material or multiple materials and may include one or more plys or layers. According to non-limiting example embodiments, the straps may include a flexible and/or stretchable portion 9, which is made e.g., of a stretchable fabric. The sides of the support may be formed from a fabric, such as a light-weight breathable, fabric, which may be an elastic material.

As indicated above, according to example embodiments, the understrap(s) and overstrap(s) may be attached to the posterior panel(s) in a detachable manner, e.g., using clips, snaps, hooks, zipper, Velcro or other attachments for detachably attaching e.g., fabric to other fabric. According to non-limiting example embodiments, at least one understrap may be detachably attached to a first posterior panel, and the at least one overstrap may be detachably attached to said second posterior panel.

While the overstraps and understraps are not generally limited in their size and shape, those skilled in the art would be able to determine an appropriate size and shape depending on the intended use of the devices and the person to whom the device is to be applied. Although example embodiments provided herein depict the understraps and overstraps in curved shapes (see, e.g., FIGS. 1-9), the straps may be formed into any desired shape that achieves the desired results discussed herein. Additionally, the size of the overstraps and understraps may be determined based on various factors including for example the size of the intended user of the device. For example, devices of the present invention may be formed into sizes such as Small, Medium, Large and Extra large, or into numerical sizes, which may call for differently sized or shaped straps. For smaller users, the straps may adapted to be shorter and narrower for example, and the curvature of the straps may be greater in comparison for example to devices adapted for larger users, in which the straps may be longer and/or thicker and the curvature may be more gradual.

According to non-limiting example embodiments, the first and second posterior panels are indirectly or directly connected or attached to one another, but may or may not come into direct contact with one another. By way of example, as depicted in FIGS. 1-5, the posterior panels may be directly or indirectly connected to one another for example with e.g., cinch strings 8. The connection may be flexible and optionally elastomeric. It is also contemplated that the connection may be adapted to be adjustable to a desired tightness/fit, e.g., by use of the tensioner reel described herein.

According to non-limiting example embodiments, the device includes four adjustable Velcro® support straps (two upperstraps and two lowerstraps) that are adjustable and can be adjusted snugly against the abdomen. Thus, these embodiments include two understraps (an upper and a lower understrap), and two corresponding overstraps (an upper and a lower overstrap) that when closed cover at least a portion of the understraps. In example embodiments, the understraps each include a portion of Velcro® over the top of the understraps (when the understraps are in a closed position). The overstraps each include the corresponding portion of Velcro® under the bottom of the overstraps (when the overstraps are in a closed position), such that the overstraps attach to the understraps.

According to any of the present example embodiments, the overstraps and understraps may be constructed such that when they are in a closed position over a user of the device, they may overlap somewhat or work in conjunction with one another such that the device may be fixed in a closed position. According to example embodiments, at least one of the understrap and the overstrap may have an attachment incorporated therein or thereon, such that when the understrap and the overstrap are in a closed position, they may be attached to one another. By way of non-limiting example, the attachment may be a hook and loop fastener attachment (e.g., Velcro®), in which the understrap may have a first portion of the hook and loop fastener (e.g., the hook portion) attached thereto, for example sewn on the understrap or attached thereto e.g., by adhesive; and the overstrap may have a second portion of the hook and loop fastener (e.g., the loop portion) attached thereto. In alternative embodiments, the attachment may be for example snaps, zippers, buttons, hook and eye, buckles, tapes or other adhesives, or other attachment known to those skilled in the art. It is noted that while the overstraps and understraps have names that imply that the overstraps are physically positioned over the understraps when the straps are in a closed position, according to alternative embodiments the straps may overlap with one another only minimally or not at all, for example in embodiments where a hook and eye attachment is utilized.

It should also be noted that the present embodiments are not limited with respect to which side the understraps or overstraps are on, e.g., right or left with respect to the wearer. Additional embodiments are envisioned in which one or more understraps are one side of a wearer, and one or more understraps are on another side of a wearer.

The posterior panel(s) are not limited by its material. According to non-limiting example embodiments the posterior panel(s) may be made of a material that will conform to the lumbar region of the person to whom the device is to be applied. Example panels may include one or more materials, such as cloth material, which may be lightweight for comfort and/or one more stiffer or reinforced materials for added stability. Example support materials that may be included for example in the posterior panel may include inner light weight (cloth) with outer-laced mesh to maximize comfort and breathability while at the same time maintaining support. The posterior panel(s) may in some embodiments include an elastic fiber strands within the material which allow for a form fit with tensioning. A central region of a panel may be made of a breathable cloth fabric to interconnect for example spandex/elastic flexible regions.

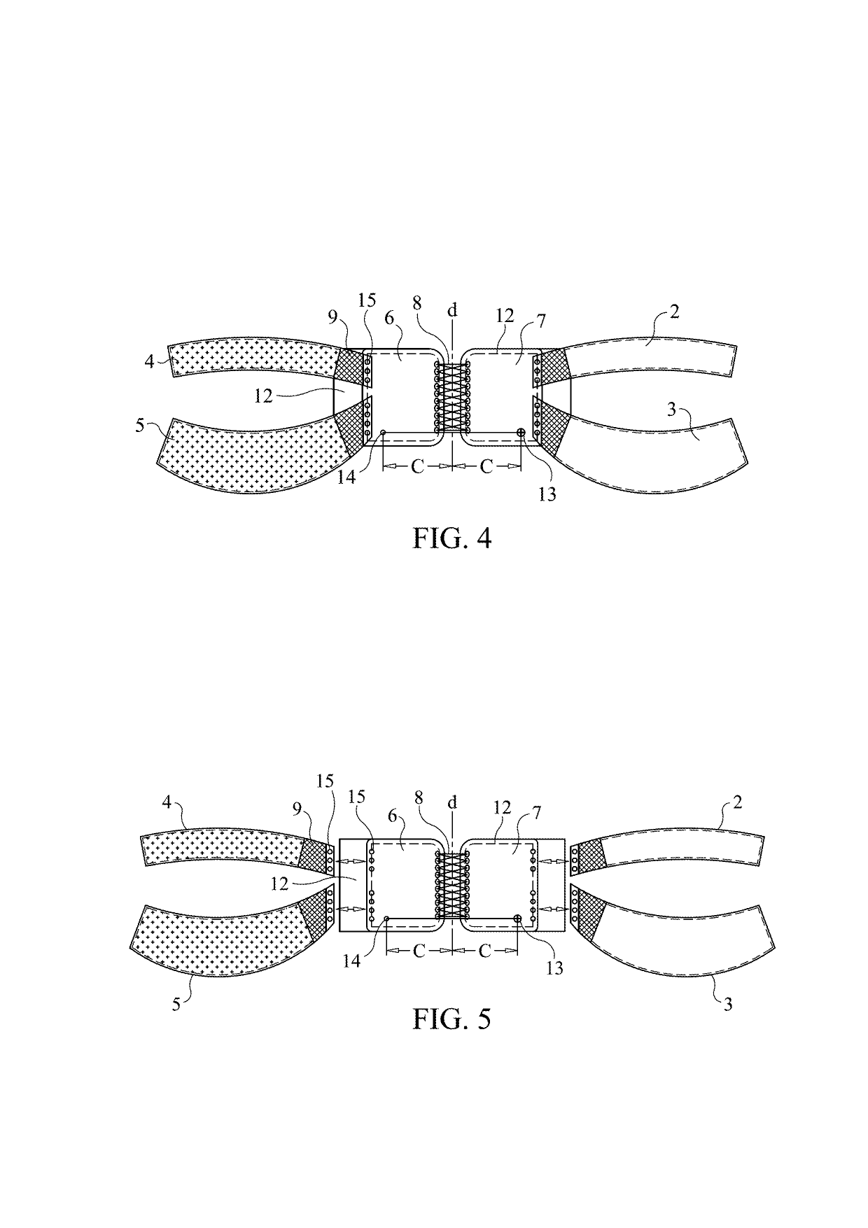

Although example embodiments provided herein depict the posterior panels in particular shapes (see, e.g., FIGS. 4 and 5), the posterior panels may include curved portions, angles, and/or any other desired shape that achieves the desired results. Additionally, the size of the posterior panels may be determined based on various factors including for example the size of the intended user of the device. For example, as indicated above, devices of the present invention may be formed into sizes such as Small, Medium, Large and Extra large, or into numerical sizes, which may call for a different size back panels.

The lumbar support device may further include an inner lining that may be attached to the posterior panels, e.g., by being sewn to the posterior panel(s). The inner lining may be made of a material such as a breathable, lightweight material, that may optionally include a stretchy material, such as spandex. The lining may be included e.g., for comfort and fit of the device as the lining would be on the inside of the device with respect to a wearer of the device. The lining may also be included to help the device maintain its shape. According to example embodiment, the lining may wrap all the way around the device and include a front/anterior portion that may be configured to fit across the front of a wearer of the device, such that the straps would wrap across the front of the lining when the straps are in a closed position. The lining may also have one or more portions that may be unzipped by a zipper 16 or unhooked or otherwise opened or loosened for ease of putting the device on or off.

Example anterior portions of a lining or anterior panels may be constructed of any desirable material and/or shape depending on the intended use of the device. For example, a front/anterior lining portion or panel may be of a shape, size and material to be able to cover an expanding pregnancy belly. By way of non-limiting example, the front panel may be made of a stretchable fabric, such as a spandex type fabric, including for example, Lycra spandex. The front panel may also include one or more moveable and/or removable portions, such that a wearer of the device may for example gather the panel under or over the wearer's belly, or remove the panel or a portion thereof from the device (e.g., on hot clays or to help conform the device to the woman's belly as it grows larger, or depending on wardrobe considerations).

According to non-limiting example embodiments, support devices may be provided without the straps, which include at least first and second posterior panels permanently attached to one another by cinch strings, that may be adjusted by a tension reel on one of the panels. These strings are fixed at an approximately equal distance on the posterior panels. The posterior panels may be fixed (e.g., sewn) to a spandex/elastic anterior/lining material, which may be adapted to wrap all the way around the belly of a wearer of the device. As with other embodiments, a tensioner reel (adjuster) may be attached to a posterior panel to allow for the cinch strings to be loosened or tightened.

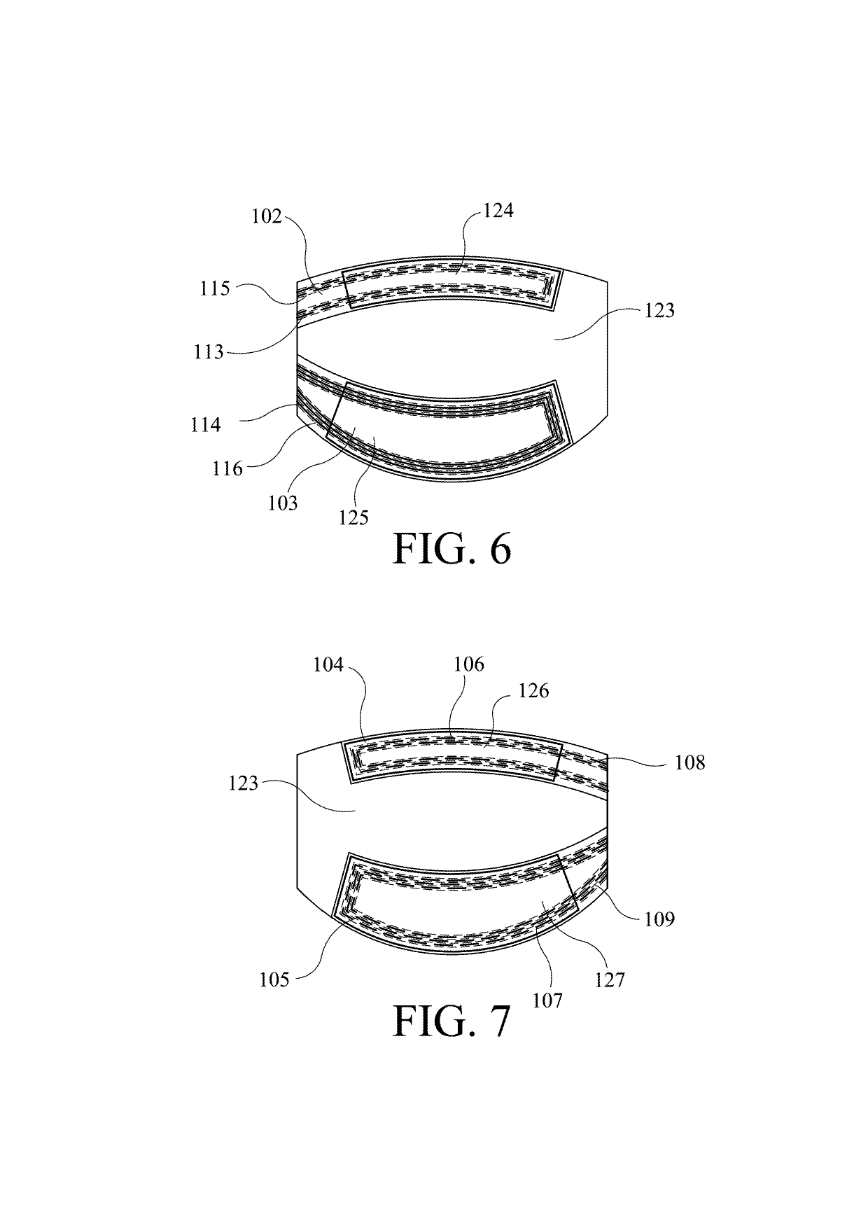

FIGS. 6-13 depict different non-limiting example embodiments of an example adjustable multi-strap lumbar support device (101) within the scope of the present invention. Referring to FIG. 6 for example, according to example embodiments, an understrap 102 may be provided, which optionally includes two channels 115 running continuously essentially the length of an understrap and back. Example embodiments are not limited with respect to the exact configuration of the channels depicted in FIG. 6. In this non-limiting example a first pullstring 113 is incorporated into one of the channels and a further pullstring (not numbered) is incorporated into a further channel (not numbered). As indicated above, the device may include a second or more understrap e.g., the depicted lower understrap (103), which may also include one or more channels 116 (three channels are depicted in the bottom understrap 103 of FIG. 6) incorporating one or more pullstrings 114 therein.

According to non-limiting example embodiments, the at least one understrap has at least a first channel formed therein, and at least a first pullstring within the first channel. According to example embodiments, two or more understraps may be provided, each having at least one channel therein, which channels are adapted to each include at least one pullstring therein.

The term “continuously” as used herein, is intended to mean that the channels are formed such that a pullstring may run through a channel uninterrupted, such that the pullstring is capable of being moved within the channel and may be pulled on or adjusted when the device is in use. Example channels may include for example channels in fabric or other material that is part of, within, over, or under a strap or panel of the device. The channel may be formed for example by stitching a channel in a two ply or more material, such as the material that makes up the straps and/or posterior panel through which the pullstring runs. Such stitching may be for example heavy duty stitching. The channels may include segments of stitching or segments of material that are not necessarily continuous without break, so long as they are adapted to allow a pullstring to run continuously through the strap and posterior panel.

According to non-limiting example embodiments, the at least one overstrap has at least one channel (a second channel) formed therein, and at least one pullstring (a second pullstring) within the second channel. According to example embodiments, two or more overstraps may be provided, each having at least one channel therein, which channels are adapted to each include at least one pullstring therein.

Referring to FIG. 7 for example, according to example embodiments an overstrap 104 may include e.g., two channels 108 running continuously essentially the length of an understrap and back. Example embodiments are not limited with respect to the exact configuration of the channels depicted in FIG. 7. In this non-limiting example a first pullstring 106 is incorporated into one of those channels and a further pullstring is incorporated into any further channels. As indicated above, the device may include a second or more overstrap 105, which may also include one or more channels 109 (three channels are depicted in the bottom overstrap of FIG. 7) incorporating one or more pullstrings 107 therein.

According to non-limiting example embodiments, one or more of the pullstrings provided in the present devices (which are present through the understraps and overstraps, travel around the back part of the support, and criss-cross in a posterior panel), may be formed from e.g., a stretchable or elastic material. For example, a suitable material may include Lycra (spandex fiber thread), and/or Latex.

Devices provided herein provide a posterior panel where at least one understrap (e.g., 102, 103) is attached to a first side of said posterior panel, and the overstrap (e.g., 104, 105) is attached to a second side of the posterior panel. The posterior panel is not limited by its shape or size, so long as it may accommodate channels that contain pullstrings as described herein, and permit a user/wearer of the device to adjust the fit of the device by adjusting the pullstrings.

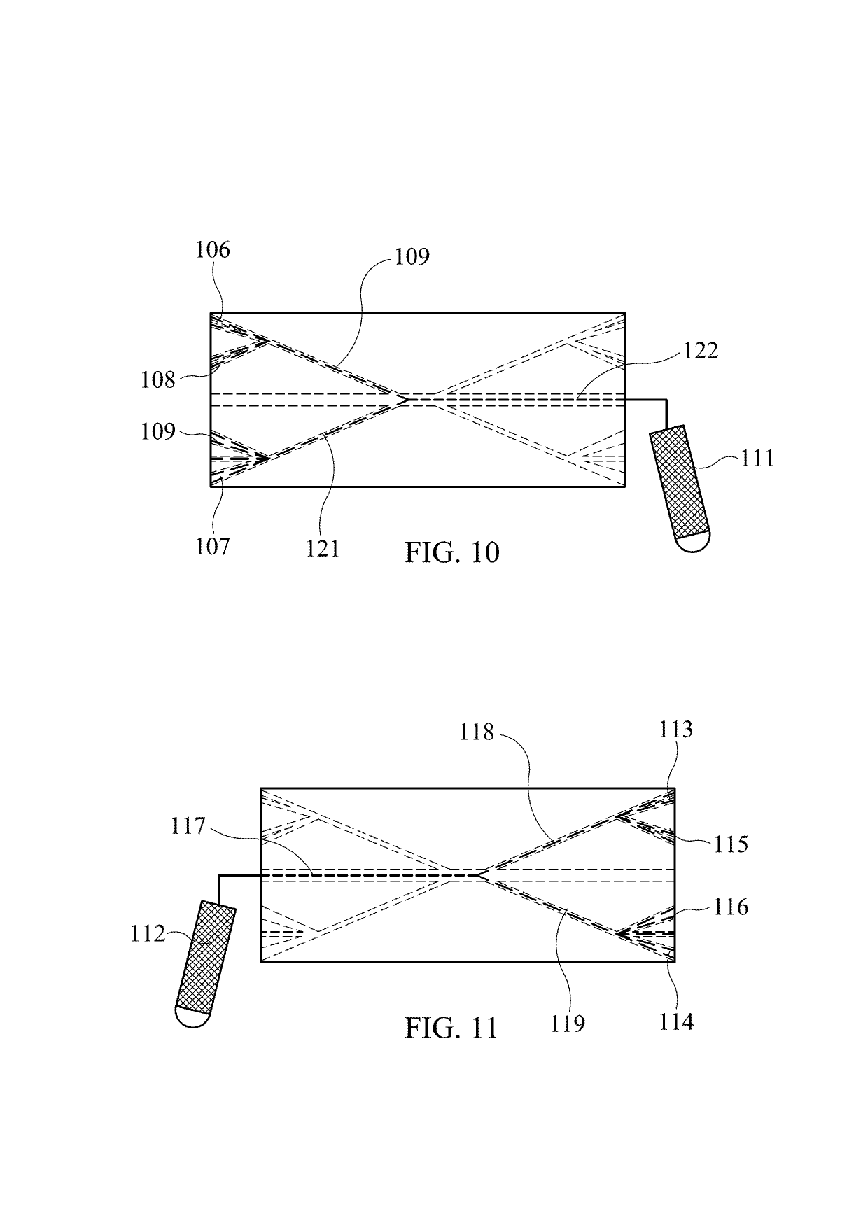

Although example embodiments provided herein depict the posterior panel in a substantially rectangular shape (see, e.g., FIGS. 10 and 11), the posterior panel may include curved portions, angles, and/or any other desired shape that achieves the desired results. Additionally, the size of the posterior panel may be determined based on various factors including for example the size of the intended user of the device. For example, as indicated above, devices of the present invention may be formed into sizes such as Small, Medium, Large and Extra large, or into numerical sizes, which may call for a different size back panel.

In example embodiments the posterior panel includes at least a third channel which is in communication with said first channel such that the first pullstring is incorporated continuously within said first channel and said third channel and crosses said posterior panel in said third channel, exiting said posterior panel on a side of said posterior panel that is opposite from the understrap. FIG. 11 for example, depicts a posterior panel having multiple channels 117, 118 and 119, which are in continuous communication with channels that extend through upper and lower understraps, such that the pullstrings (e.g., 113 and 114) that extend through the upper and lower understraps continuously extend across at least a portion of the posterior panel.

The posterior panel may include at least a fourth channel which is in communication with the second channel such that the second pullstring is incorporated continuously within said second channel and said fourth channel and crosses said posterior panel in said fourth channel, exiting said posterior panel on a side of said posterior panel that is opposite from the overstrap. FIG. 10 for example, depicts a posterior panel having multiple channels 120, 121 and 122, which are in continuous communication with channels that extend through upper and lower overstraps, such that the pullstrings that extend through the upper and lower overstraps continuously extend across at least a portion of the posterior panel.

In example embodiments, the pullstring(s) extend across at least 50% of the posterior panel and the pullstring(s) exit the panel from an opposite side of posterior panel from the strap that includes those pullstrings. The pullstring may exit the panel for example on the side of a person using the device to allow for ease of adjustment and comfort while doing so.

According to example embodiments, the pullstrings terminate/connect to one or more pullstring attachments. By way of non-limiting example embodiment, the pullstring attachments may be adjustable pull-string straps having for example, pull rings, such as plastic or metal pull rings thereon. By way of further example, the pullstring attachment may include girdle-like interconnections of elastic fibers. According to example embodiments, devices may include a first pullstring attachment on a portion of the first pullstring that has exited the posterior panel. The pullstring attachment may attached to the first pullstring and/or additional pullstrings by any appropriate method. The pullstring attachment may be adapted such that a user of the device may adjust a fit of the understrap by adjusting the first pullstring and/or any additional pullstrings incorporated in said at least one understrap, using said the pullstring attachment, e.g., by pulling pullstring attachment anteriorly. (See for example, pullstring attachment 112 in FIG. 11 and other figures).

According to example embodiments, devices may include a second pullstring attachment on a portion of the second pullstring that has exited the posterior panel. The pullstring attachment may attached to the second pullstring and/or additional pullstrings by any appropriate method. The pullstring attachment may be adapted such that a user of the device may adjust a fit of the overstrap by adjusting the second pullstring and/or any additional pullstrings incorporated in said at least one overstrap, using the pullstring attachment, e.g., by pulling pullstring attachment anteriorly. (See for example, pullstring attachment 111 in FIG. 10 and other figures).

The pullstring attachments may be of any suitable size or shape and may optionally themselves include an attachment, such that they may be attached to another portion of the device, rather than hanging down loose from the device.

Devices herein may also include one or more front/anterior panels (see e.g. front panel 123 on FIG. 6). Example anterior panels may be constructed of any desirable material and/or shape depending on the intended use of the device. For example, a front/anterior panel may be of a shape, size and material to be able to cover an expanding pregnancy belly. By way of non-limiting example, the front panel may be made of a stretchable fabric, such as a spandex type fabric, including for example, Lycra spandex. The front panel may also include one or more moveable and/or removable portions, such that a wearer of the device may for example gather the panel under or over the wearer's belly, or remove the panel or a portion thereof from the device (e.g., on hot days or to help conform the device to the woman's belly as it grows larger, or depending on wardrobe considerations).

According to non-limiting examples of these embodiments, the device includes four adjustable Velcro® support straps (two upperstraps and two lowerstraps) that are adjustable and can be used adjust snugly against the abdomen. Thus, these embodiments include two understraps (an upper and a lower understrap), and two corresponding overstraps (an upper and a lower overstrap) that when closed cover at least a portion of the understraps. In example embodiments, the understraps each include a portion of Velcro® over the top of the understraps (when the understraps are in a closed position). The overstraps each include the corresponding portion of Velcro® under the bottom of the overstraps (when the overstraps are in a closed position), such that the overstraps attach to the understraps. These embodiments may further include pull strings throughout the device that are made of an elastic material. Once the adjustable Velcro® straps are snug to the abdomen/belly, adjustable pull string support straps will be pulled anteriorly (toward the front of the woman) in order to pull the support snug (and help it conform the belly and back). These pull straps may have two pull strings that allow the user to conform the support to their bodies' natural contours (posteriorly and anteriorly) (both front and back). In such embodiments, a support uses both Velcro® straps and elastic pull strings within the straps to allow the support to be adjusted to naturally contour/conform e.g., to a pregnant woman's anatomy, which changes throughout pregnancy. The elastic pull strings within the straps in these embodiments, allow the support to safely conform to the woman's anatomy and avoid over tensioning because they are elastic.

As discussed above with respect to other non-limiting example embodiments, the overstraps and understraps in these embodiments may have an attachment incorporated therein or thereon, such that when the understrap and the overstrap are in a closed position, they may be attached to one another. By way of non-limiting example, the attachment may be a hook and loop fastener attachment (e.g., Velcro®), in which the understrap may have a first portion of the hook and loop fastener (e.g., the hook portion) attached thereto, for example sewn on the understrap or attached thereto by adhesive or other; and the overstrap may have a second portion of the hook and loop fastener (e.g., the loop portion) attached thereto. In alternative embodiments, the attachment may be for example snaps, zippers, buttons, hook and eye, buckles, tapes or other adhesives, or other attachment known to those skilled in the art.

The posterior panel or panels are also not limited by its material. According to non-limiting example embodiments the posterior panel(s) may be made of a material that will conform to the lumbar region of the person to whom the device is to be applied. Example panels may include one or more materials, such as cloth material, which may be lightweight for comfort and/or one more stiffer or reinforced materials for added stability. Example support materials that may be included for example in the posterior panel may include inner light weight (cloth) with outer-laced mesh to maximize comfort and breathability while at the same time maintaining support. The posterior panel(s) may in some embodiments include an elastic fiber strands within the material which allow for a form fit with tensioning. A central region of a panel may be made of a breathable cloth fabric to interconnect for example spandex/elastic flexible regions.

According example embodiments, sides of the support may include a portion of the posterior panel and/or a portion of the front panel, or may be separate side portions of the support, which are attached to both the posterior and anterior panels. In embodiments having a separate side panel, the pull string may continue through one or more channel in the side panel and may exit the device at the side panel, rather than from the posterior panel. Also, in embodiments having a separate side panel, the straps may be attached to the device at the front panel, side panel and/or at the posterior panel. The sides of the support may be formed from a fabric, such as a light-weight breathable, fabric, which may be an elastic material.

Also provided herein are methods of preventing or treating back pain in a person (such as a pregnant, human female or other person who may benefit from support provided by wearing the device) that include applying the devices described herein to a person, such that the person may wear the device. Examples of such devices may include e.g., the devices described herein.

According to non-limiting example embodiments, the support may be used for specified periods of time. According to non-limiting example embodiments, the present support devices may be used for example on a pregnant, human female throughout a woman's pregnancy, e.g., daily (e.g., 8 to 10 hours a day, 5 to 7 days a week), weekly (for example, several hours a day, one or two days a week), periodically (e.g., for four hours every morning or evening, or for increasing periods of time as the pregnancy progresses) or occasionally (for example during a portion of the day, when at work, when standing, when sitting for long periods of time, e.g., at the computer, on long car rides, when leaning over for greater than five minutes, when performing house chores, when gardening, when back pain starts to arise, or during other times of excess stress on one's back). In embodiments in which the present support or brace devices are used during a woman's pregnancy, the device may be adjusted to naturally contour/conform to the woman's anatomy, which changes throughout pregnancy.

The adjustability of the present support devices maximizes the support's effectiveness because it is form fitted to the rapidly changing anatomy caused by pregnancy. Ultimately, this decreases the stresses on the spine, improving posture/alignment during activities of daily living.

According to example embodiments, the devices may be applied to a person either by the person themselves or with the assistance of another person. The devices may be applied by a method that includes positioning the device on the person such that the posterior panel(s) is positioned across a back of the person. Such positioning may include for example, stepping into one of the present devices and pulling it up to the person's waist level, or slipping the device over the person's head and sliding it down to the person's waist. Alternatively, depending on the construction of the device, the device may be adapted such that a portion of the device may be opened and closed around the waist of the person without the need for stepping into the device or putting it on over one's head. For example one or both sides of the device may be attached together, for example where a front panel and back panel meet each other or any optional side panel, e.g., with buttons, zipper, hook and eye, snaps, loop and hook (e.g., Velcro®), or another attachment. Alternatively, the lining may have an opening with attachment for closing the opening. In such embodiments, a user can simply open the attachment (if closed), wrap the device around their waist and attach the attachment, and then turn the device around one's waist (if necessary) to position the posterior panel across the back of the person. If a front panel or anterior portion of lining is present, the front panel or lining may be adjusted, e.g., any wrinkles in the material may be smoothed out, so that the front panel or lining is comfortable. Once positioned correctly, one may close the straps over the abdomen of the wearer, each understrap being closed prior to its corresponding overstrap.

By way of non-limiting example, the device may be applied to a wearer by a method comprising positioning the device on the person such that the posterior panel(s) are positioned across a back of the person; positioning the at least one understrap over an abdomen of the person; and positioning the at least one overstrap over a corresponding understrap, such that the overstrap attaches to the understrap.

In non-limiting example embodiments, it may be advantageous to close and position the lower straps before upper straps are positioned. These support straps are adjustable and can be adjusted snugly against the abdomen of the wearer of the device.

According to example embodiments in which the device includes cinch strings, example methods may further include adjusting cinch strings between said anterior panels to adjust a fit of the device.

Once appropriately adjusted, the support device will function to pull in the lumbar vertebrae, increasing lordosis and placing the center of gravity in a more natural position.

In embodiments having pullstrings, adjustable pull string attachments/support straps may be pulled anteriorly (toward the front of the wearer) for example using finger hooks in the pull string attachments, to pull the support snug (and help it conform the belly and back). When one pulls on the pullstrings, for example by pulling on a pull string attachment, the pullstring attachment may be held by the wearer of the device and pulled anteriorly (toward the front), which pulls the straps snug (and therefore allows them to have a better fit) and also pulls the posterior back component snug to the back.

The pull string strap may be attached to another portion of the device, e.g., using Velcro® or other attachment. The outside of an overstrap or visible portion of an understrap when the device is in a closed position, may include a hook and loop fastener adapted or positioned to have the pull string strap attached thereto.

In embodiments having cinch strings connecting posterior panels, the cinch strings may be adjusted (e.g., tightened or loosened) to adjust the fit of the device on the wearer.

Example embodiments provided herein are directed to kits that may include at least one lumbar support device provided herein and instructions for use of the device. Such instructions may include for example instructions regarding proper positioning and application of the device on a person and methods of adjusting the device to achieve a proper fit. Example instructions may provide guidance with respect to when and how long to wear the device to achieve the best results. Further example instructions may provide instructions regarding visiting a physician when necessary and/or exercises the user may perform even when not wearing the device to improve muscle stability, help strengthen core and/or back muscles and help prevent and/or lessen back pain by methods in addition to wearing the device. Further example instructions may include instructions for cleaning the device.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### EXPERIMENTAL EXAMPLES

### Example 1

FIGS. 1-5 depict non-limiting example embodiments of the present devices, which embodiments are adapted for use specifically on a human female. FIGS. 1-3 depict front/anterior views of example multi-strap lumbar support devices (1) provided herein. FIGS. 4-5 depict rear/posterior views of the example multi-strap support devices (1) depicted in FIGS. 1-3.

FIG. 1 shows top and bottom understraps (2 and 3, respectively) and top and bottom overstraps (4 and 5, respectively) in an open position, i.e., not closed in position over a wearer of the device, such as a pregnant female human. As can be seen in FIG. 1, the bottom straps are configured, such that they can close in the direction of arrows A across the front of the device. The overstraps are configured to be able to close in direction B across the front of the device after the understraps are already closed. Thus the overstraps in these embodiments are closed “over” the understraps. FIG. 1 also depicts examples of straps that may have more than one type of material to provide a better fit to the wearer. In the depicted example, the straps may include, e.g., a portion 9 of any suitable size or shape, which may be made of a stretchable material. For example, portion 9 may be an elastic portion of the straps to allow for easy adjustment of the straps/device over the wearer.

FIG. 1 also shows (see the dotted lines on the straps) an example of where a hook and loop fastener such as Velcro, or another fastener may be attached to the straps. By way of non-limiting example, in the embodiments depicted in FIG. 1, the “female” portion of a hook and loop fastener may be attached to the underside of the overstraps, and the “male” portion of a hook and loop fastener may be attached to a top side of the understraps, so that when the straps are folded across a wearer, the “male” and “female” portions come into contact with one another and secure the straps in a desired place/fit. It should be understood however, that the fasteners may be varied and that even when hook and loop fasteners are used, the fasteners may be reversed (e.g., male on the underside of the overstraps and “female” on the top of the understraps), or partially reversed (e.g., the top overstrap having the “female” portion, and the bottom overstrap having the “male” portion).

FIG. 2 shows the top and bottom understraps (2 and 3, respectively) after they have been positioned across the front of the device in a closed position, while the overstraps (4 and 5) remain in an open position. When the understraps are in position across the device as shown in FIG. 2, in embodiments having a hook and loop fastener, one portion (e.g., the “male” portion) of the hook and loop fastener 11 would be visible/exposed on the outside of the device as depicted in FIG. 2. The hook and loop fastener may be attached to the straps, e.g., by being stitched or adhesively attached thereto. FIG. 2 also depicts (within the dotted lines) the area of the overstraps 4 and 5 which have corresponding hook and loop fastener (e.g., the “female” portion) attached thereto (e.g., by stitching or adhesive). According to non-limiting embodiments the hook and loop fastener portions would be attached to the straps in a configuration that would allow for as much overlap as possible of male and female portions when the overstraps are folded into position over the understraps. The more overlap of the hook and loop fastener, the more secure the fit and the less of the strap may be hanging loose, which may irritate a wearer and/or cause awkward lumps or bumps when the present devices are provided under clothing.

FIG. 3 shows the top and bottom overstraps (4 and 5) in a closed position across the front of the device, over the top of corresponding top and bottom understraps. Again, the dotted outline shows the position of a “female” portion of the hook and loop fastener, which is now on the underside of the outerstraps

According to non-limiting example embodiments, an inner lining of the brace may be fabricated from light-weight, breathable and stretchable fabric, for fit and comfort of the wearer.

In example embodiments, the device includes an inner lining 12, which may extend around to the front/anterior portion of the brace. A front portion of the inner lining may be positioned over the belly of a wearer of the device prior to closing the straps into position. According to example embodiments, the inner lining may be made of a breathable, stretchable material.

FIGS. 4 and 5 depict back/posterior views of an example multi-strap lumbar support device (1) provided herein. The back/posterior view in FIGS. 4 and 5 depict two posterior panels (6 and 7), which may be provided in a different shapes and/or sizes in other example embodiments. The back aspect of the support in this embodiment may be made of a durable, breathable fabric, and may optionally include one or more spandex and/or elastic regions. It is also contemplated that the back may include more than two panels according to non-limiting example embodiments.

As depicted in FIGS. 4 and 5, the posterior panels may be connected to one another e.g. by cinch strings 8, which may be elastic, or by other suitable attachment. The depicted cinch strings further allow for providing good fit and flexibility of the device. The cinch strings may have an adjustment dial 13 positioned on one panel at a distance “c” from the center of the cinch strings, to allow for adjusting the fit of the cinch strings. There may also be a cinch string anchor set 14, which is positioned on an opposite panel at a distance “c” from the center of the cinch strings “d”, such that the adjustment dial 13 and the cinch string anchor set 14 are approximately equal distant from a center of the cinch stings “d”.

FIGS. 4 and 5 also show that the present devices may include one or more portions of inner lining 12, which as indicated above may be fabricated, e.g., from light-weight, breathable and/or stretchable fabric. The inner lining may be attached to other portions of the device, such as the back panels, e.g., by stitching. The inner lining 12 may also have at least one way of opening or loosening the inner lining for ease of putting the device on, such as the depicted zipper 16.

FIGS. 4 and 5 also show that according to the present non-limiting embodiments, one or more of the straps may be detachable from the device. In the depicted embodiments, all four of the upper and lower, understraps and overstraps are detachable by way of snaps provided on the straps and posterior panels. FIG. 4 shows the straps in an attached configuration, with snaps 15 being provided for such attachment. Although the depicted embodiment shows three snaps for each upper strap and four snaps for each lower strap, it is contemplated that other numbers, sizes and configurations of snaps may be suitable for attachment and detachment of the straps. It is also contemplated that the straps may be attached/detached using other forms of attachment, such as hook and loop attachments and other attachment methods that would be apparent to those skilled in the art. FIG. 5 shows the straps in a detached configuration, and shows corresponding snap portions 15 on each of the straps and panels that align with one another such that they can be snapped together.

Further depicted in FIGS. 4 and 5 are the lightweight stretchable sides of the device 9.

While two understraps are depicted and two overstraps are depicted in the FIGS., it is possible that there could be three or more of each strap in alternative embodiments.

It is noted that while a particular shape is shown for the present device, the device may look somewhat different when worn depending e.g., on the size of the wearer's belly. For example, if a person wearing the device is nine months pregnant or pregnant with multiples, the upper and lower straps may appear much further apart than if the wearer is four months pregnant. The straps may also be configured somewhat differently than as shown, and such embodiments would be within the scope of the present invention.

### Example 2

FIGS. 6-13 depict a different non-limiting example embodiment of the present devices, which embodiment is adapted for use specifically on a human female. FIGS. 6 and 7 depict front/anterior views of an example multi-strap lumbar support device provided herein. FIG. 6 shows the top and bottom understraps (102 and 103) across the front of the device. FIG. 7 shows the top and bottom overstraps (104 and 105) across the front of the device. The in this example straps may be made of a breathable cloth material.

In this example the understraps are depicted as being on the right side of the wearer of the support and the overstraps on the left. But other embodiments are possible for example, in which the overstraps are on the right side and the understraps are on the left, so long as the attachment (e.g., loop and hook (Velcro®), buttons, zipper, hook and eye, snaps, etc.) are arranged on the correct side (e.g., top or bottom) and position of each strap such that the overstraps and understraps may be attached to one another.

Additionally, it is possible that the understrap and/or a portion of the overstrap can be adapted such that it can attach to the front panel of the device, (e.g., using buttons, hook and loop, etc.), or to the person wearing the device (e.g., using adhesive), in a case where the front panel or a portion thereof is removed or otherwise not present.

As depicted in FIGS. 6 and 7, the attachment in these embodiments for attaching the understrap and overstrap may be loop and hook (Velcro®). FIG. 6 shows that either the loop or hook portion of the attachment is sewn onto each of the upper understrap 124 and the lower understrap 125. FIG. 7 shows that the opposite loop or hook portion of the attachment (from the portion sewn onto the understraps), is sewn onto each of the upper overstrap 126 and the lower overstrap 127.

While two understraps are depicted and two overstraps are depicted in the FIGS., it is possible that there could be three or more of each strap in alternative embodiments.

It is noted that while a particular shape is shown for the present device, the device may look somewhat different when worn depending e.g., on the size of the wearer's belly. For example, if a person wearing the device is nine months pregnant or pregnant with multiples, the upper and lower straps may appear much further apart than if the wearer is four months pregnant. The straps may also be configured somewhat differently than as shown, and such embodiments would be within the scope of the present invention.

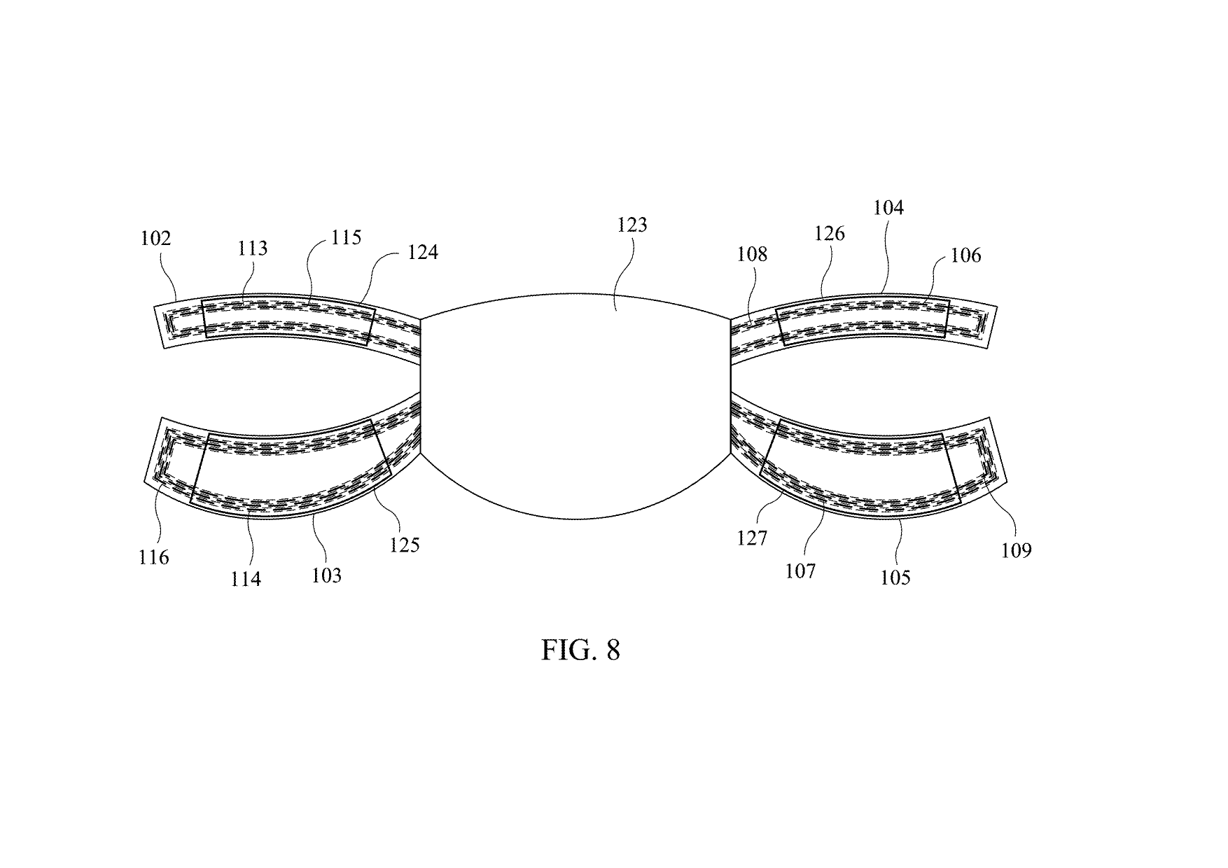

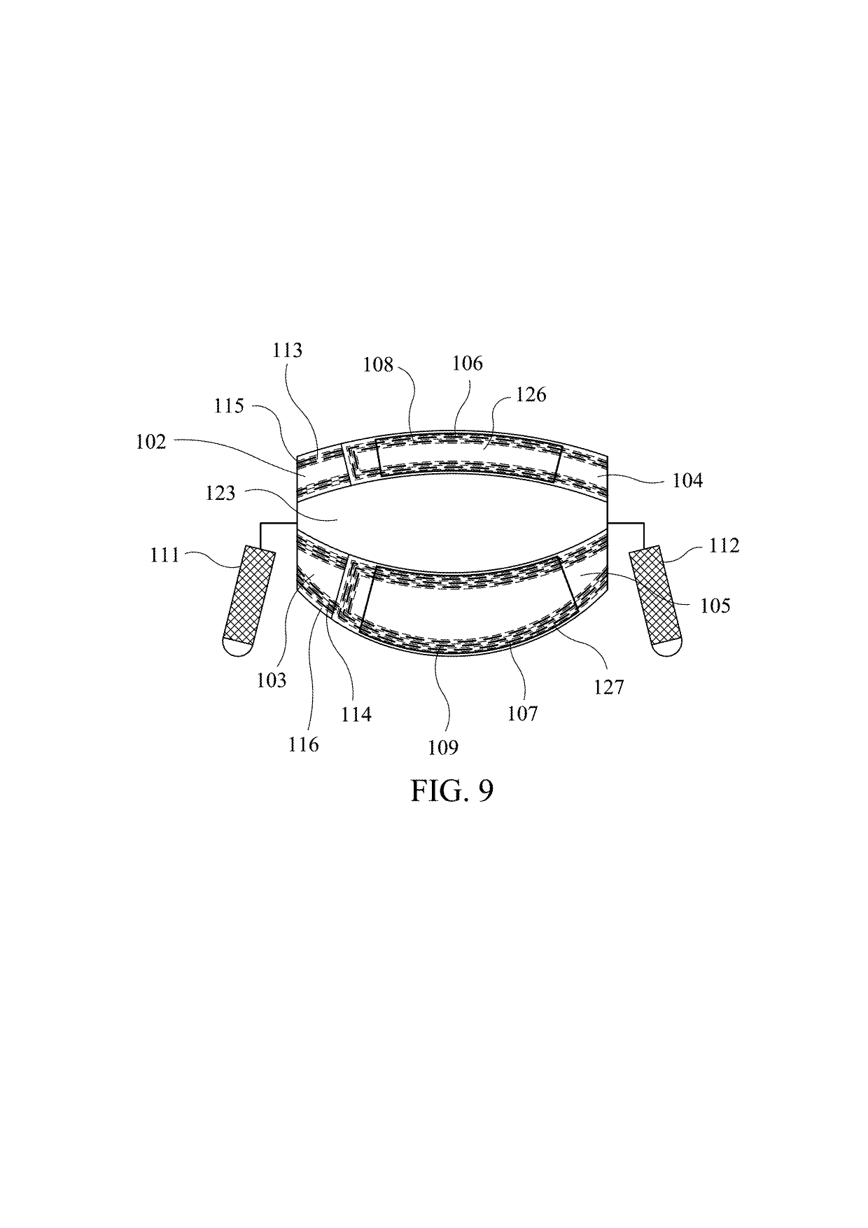

FIG. 8 depicts a front/anterior view of the example multi-strap lumbar support device. This view depicts the top and bottom understraps (102 and 103) and the top and bottom overstraps (104 and 105) open and not in a closed position over a wearer of the device. As shown in FIG. 8, the portion of the attachment (124 and 125) sewn onto the top and bottom understraps are behind the understraps (102 and 103) when the device is open, and the other portion of the attachment (126 and 127) on top of the overstraps (104 and 105) when the device is opened, so that when the device is closed (understraps then overstraps) as shown in FIG. 9, the hook and loop portions will contact each other, thus attaching the overstraps to the understraps.

As described further below, the overstraps and understraps all have pullstrings incorporated therein that travel around the back part of the support that cross one another and exit the device on the opposite side, such that a user may pull on the pullstrings (e.g., via a pullstring attachment) to adjust the fit of the overstraps and understraps).

In example embodiments, a user of the depicted example device may initially either put the device over their head, or step into it or (not shown) open one or more portions of the device e.g., by zipper. Then the user may position the posterior panel (depicted e.g., in FIGS. 10 and 11) at the user's lower back, and position an anterior, front panel 123 over their belly. The anterior, front panel may be adjusted to properly position it over the belly, for example to smooth out any wrinkles. While the depicted embodiments show a front panel, it is noted that example embodiments may not include a front panel, or the front panel or portions thereof may be removable. In embodiments that include a front panel, the front panel may be wholly or partially made out of an elastic, spandex-type material.

The user would adjust at least one of the understraps (102 or 103) over the front of the user and close the corresponding overstrap(s) (104 or 105) over the top of at least a portion of the understrap, such that the overstrap attaches to the understrap, e.g., using hook and loop attachment. According to the depicted example embodiment, the lower understrap 103 is first positioned across the user's belly and then the lower overstrap 105 is comfortably positioned over the top of the lower understrap 103 to conform to a lower portion of the belly. The upper understrap 102 is positioned across the user's belly and then the upper overstrap 104 is comfortably positioned over the top of the upper overstrap to conform to an upper portion of the belly.

FIG. 9 depicts a front/anterior view of an example multi-strap lumbar support device provided herein. This view depicts the top and bottom understraps (102 and 103) in a closed position across the front of the device, with the top and bottom overstraps (104 and 105) in a closed position over the top of the respective top and bottom understraps.

FIGS. 10 and 11 depict back/posterior views of an example multi-strap lumbar support device provided herein. The back/posterior view in FIGS. 10 and 11 depicts a rectangular posterior panel, which (as discussed above) may be in a different shape in other example embodiments. The back aspect of the support in this embodiment may be made of an elastic central region with breathable cloth fabric to interconnect spandex/elastic flexible regions of the device.

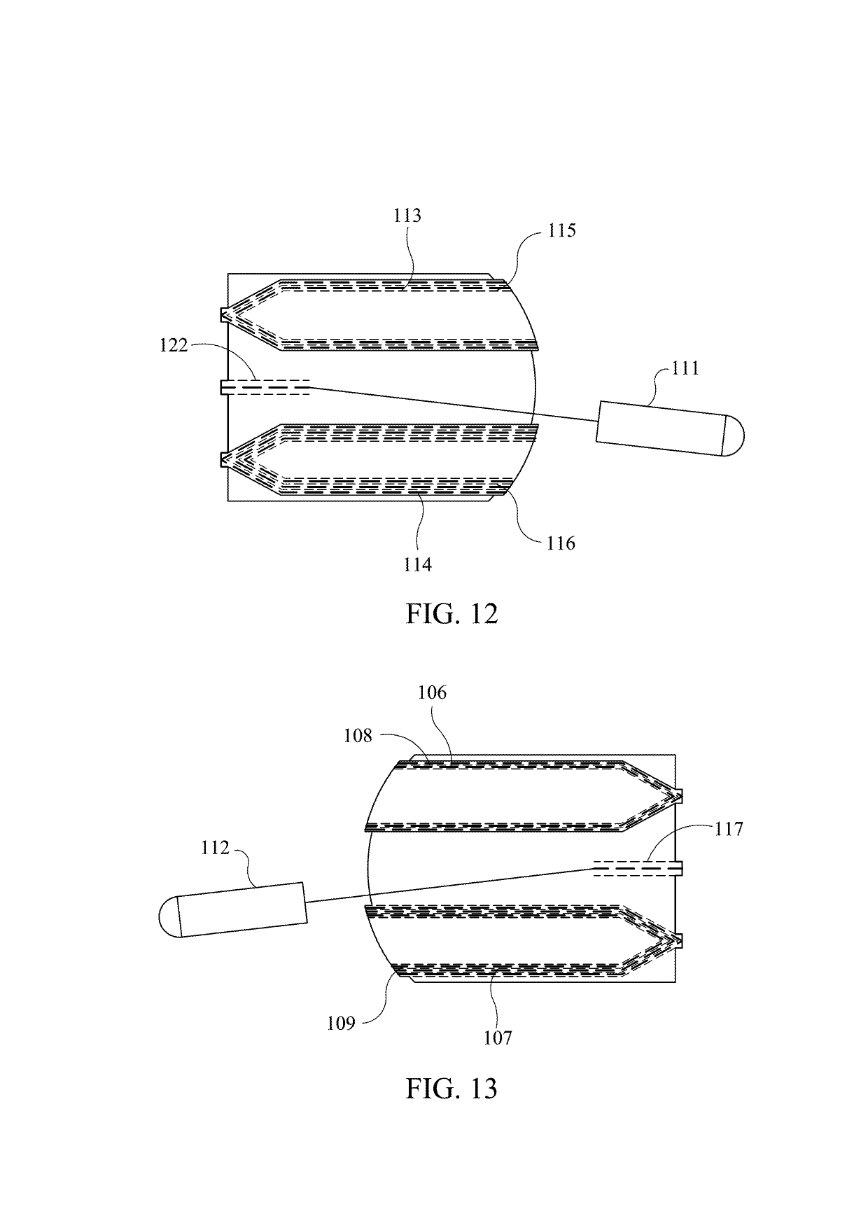

As shown in FIGS. 7, 8, 9, 10 and 13, multiple pullstrings (e.g., 106 and 107) from multiple channels (e.g., 108 and 109) may be provided through the upper and lower overstraps (104 and 105) on the anterior side of the device, which may be consolidated into one or more pullstrings through for example, a decreasing number of channels (e.g., channels 120 and 121), and eventually a single channel 122 in the posterior panel of the device (see FIGS. 10 and 12). According to the depicted examples, the pullstrings may comprise an elastic material. The consolidated pullstrings (e.g., 106 and 107) may attach to a pullstring attachment 111, such that a user/wearer of the device may pull the pullstrings using the pullstring attachment 111 to adjust the fit of the device.

As shown in FIGS. 6, and 8-11 multiple pullstrings (e.g., 113 and 114) from multiple channels (e.g., 115 and 116) are provided through the upper and lower understraps on the anterior side of the device, which may be consolidated into one or more pullstrings through for example, a decreasing number of channels (e.g., channels 118 and 119), and eventually a single channel 117 in the posterior panel of the device (see FIGS. 11 and 13). According to the depicted examples, the pullstrings may comprise an elastic material. The consolidated pullstrings (e.g., 113 and 114) may attach to a pullstring attachment 112, such that a user/wearer of the device may pull the pullstrings using the pullstring attachment 112 to adjust the fit of the device.

The pullstring attachments may include for example finger hooks that may assist the user in gripping the pullstring attachment and/or pulling on the pullstring attachment.

According to the depicted examples, channels may be formed in various portions of the device (such as the under and overstraps and the posterior panel) by forming those portions of the device using two or more plys of material that are stitched to form channels. Such a formation, may provide more comfort and a smoother device than if channels are added by way of adding additional material e.g., to the outside or the inside of the primary material of each strap or panel.

Although for clarity, each of FIGS. 10 and 11 shows the pullstrings from panels on one side of the device going through the back of the device to a pullstring attachment on the opposite side of the device, both sets of pullstrings from all panels are actually present in the device at the same time and cross one another in the posterior panel of the device.

FIG. 12 depicts a right side view of an example multi-strap lumbar support device in position over a human. As shown in FIG. 12, the pullstrings may be spread over various positions of the human's belly. When the device is being used by a woman that is only four months pregnant the straps may cover a larger portion of her belly, and various drawstrings may span over a greater percentage of her belly than when she is nine months pregnant. As shown in FIG. 12, the pullstrings are consolidated toward the back of the device and may be adjusted by pulling on a pullstring attachment on the other side of the human. That is, FIG. 12 depicts pullstrings (e.g., 113 and 114) throughout the right side (understraps) of the device, and the shown consolidated pullstring 122, which attaches to pullstring attachment 111 may be pulled or loosened to adjust the fit of the pullstrings in the opposite/left side (overstraps) of the device.

FIG. 13 depicts a left side view of an example multi-strap lumbar support device in position over a human. As shown in FIG. 13, the pullstrings may be spread over various positions of the human's belly, but are consolidated toward the back and may be adjusted by pulling on a pullstring attachment on the other side of the human. That is, FIG. 13 depicts pullstrings (e.g., 106 and 107) throughout the left side (overstraps) of the device, but the shown consolidated pullstring 117, which attaches to pullstring attachment 112 may be pulled or loosened to adjust the fit of the pullstrings in the opposite/right side (understraps) of the device.

After the understraps and overstraps are positioned over a person's abdomen and attached to one another by attachment, then the wearer may adjust the device by adjusting the pullstrings. The pullstrings may be adjusted for example by pulling on the respective pullstring attachments 111 and 112, e.g., anteriorly (toward the front), which pulls on the pullstrings and therefore, tighten the straps over the front of the body to help it conform to the belly and back.

In the event that one wishes to loosen the device, the pullstrings may be loosened either before putting the device on the user/wearer or after it is in position over the user/wearer.

In non-limiting example embodiments, one or more of the pullstring attachments may then be attached to the device, e.g., to an anterior panel, to a side panel or to the posterior panel so they do not simply hang down and potentially either cause the belt to not be secure, or show through the clothing worn over the top of the belt. Such attachment may also be for example by loop and hook (Velcro®), buttons, zipper, hook and eye, snaps, etc, and the attachment for attaching the pull string attachment to another portion of the device need not be the same as the attachment for attaching the overstraps and understraps to one another.

According to this example embodiment, the sides of the support may include a portion of the posterior panel and/or a portion of the front panel, or may be separate side portions of the support. The sides of the support may be formed from a fabric, such as a light-weight breathable, fabric, which may be an elastic material.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such modifications fall within the scope of the present invention as defined by the claims appended hereto.

What is claimed is:

1. A lumbar support device comprising
at least one understrap;
at least one overstrap; and
at least first and second posterior panels, said at least one understrap being attached to said first posterior panel, and said at least one overstrap being attached to said second posterior panel;
wherein said first and second posterior panels are adjustably connected to one another; and
wherein said at least one understrap and said at least one overstrap are configured to be attachable to one another when said understrap and said overstrap are in a closed position.

2. The lumbar support device of claim 1, wherein said first and second posterior panels are connected to one another with cinch strings.

3. The lumbar support device of claim 1, further comprising an inner lining.

4. The lumbar support device of claim 1, wherein said at least one understrap is detachable from said first posterior panel, and said at least one overstrap is detachable from said second posterior panel.

5. The lumbar support device of claim 1, wherein the at least one understrap and the at least one overstrap are attachable to one another by hook and loop fastener.

6. The lumbar support device of claim 1, wherein said device comprises at least two understraps and at least two overstraps.

7. A kit comprising
(1) lumbar support device of claim 1; and
(2) instructions for using the lumbar support device.

8. The kit of claim 7, wherein said instructions include instructions regarding proper application, positioning and adjustment of the device on a person.

9. A lumbar support device comprising
at least one understrap having at least a first channel formed therein, and having at least a first pullstring within said first channel;
at least one overstrap having at least a second channel formed therein, and having at least a second pullstring within said second channel;
a posterior panel having said understrap attached thereto on a first side of said posterior panel, and having said overstrap attached to a second side of said posterior panel;
wherein said posterior panel comprises at least a third channel which is in communication with said first channel such that the first pullstring is incorporated continuously within said first channel and said third channel and crosses said posterior panel in said third channel, exiting said posterior panel on a side of said posterior panel that is opposite from the understrap;
wherein said posterior panel comprises at least a fourth channel which is in communication with said second channel such that a second pullstring is incorporated continuously within said second channel and said fourth channel and crosses said posterior panel in said fourth channel, exiting said posterior panel on a side of said posterior panel that is opposite from the overstrap; and
wherein at least one of said at least one understrap and said at least one overstrap has an attachment incorporated thereon, such that when said understrap and said overstrap are in a closed position, the overstrap may be attached to the understrap.

10. The device of claim 9, further comprising a first pullstring attachment on a portion of said first pullstring that has exited said posterior panel, adapted such that a user of the device may adjust a fit of said understrap by adjusting said first pullstring attachment.

11. The device of claim 9, further comprising a second pullstring attachment on a portion of said second pullstring that has exited said posterior panel, adapted such that a user of the device may adjust a fit of said overstrap by adjusting said second pullstring attachment.

12. The device of claim 9, further comprising at least one anterior panel attached to said posterior panel.

13. The device of claim 10, further comprising at least one attachment adapted to attach the first pullstring attachment to an anterior panel of the device; wherein said anterior panel is directly or indirectly attached to said posterior panel.

14. The device of claim 13, further comprising at least one attachment adapted to attach the second pullstring attachment to an anterior panel of the device; wherein said anterior panel is directly or indirectly attached to said posterior panel.

15. A method of preventing or treating hack pain in a person, comprising applying the lumbar support device of claim 9 to the person.

16. A kit comprising
(1) the lumbar support of claim 9; and
(2) instructions for using the lumbar support device.

17. A lumbar support device comprising
first and second posterior panels connected to each other by adjustable cinch strings; and
an inner lining attached to said posterior panels, configured to wrap around the abdomen and back of a person wearing the device, and support the belly of the person.

18. The lumbar support device of claim 17, further comprising at least one understrap configured to attach to and detach from at least one posterior panel; and at least one overstrap configured to attach to and detach from at least one posterior panel; wherein the understrap is also configured to wrap across the front of the abdomen of the person, and the overstrap is configured to wrap at least partially over and attach to the understrap.
